(19) United States
(12) Patent Application Publication
(10) Pub. No.: US 2024/0130666 A1
(43) Pub. Date: Apr. 25, 2024

(54) Electronic Monitoring Device called HELU which Measures Body Metrics like Pulse, Heart Rates, and Blood Pressure at Rest, and which creates a well-defined performance measurement that is used to monitor, analyze, and optimize all characteristics of Sexual Activity and Erectile Dysfunction.

(71) Applicants: James Spear, Pensacola, FL (US); Josh Wells, Carmel, IN (US)

(72) Inventors: James Spear, Pensacola, FL (US); Josh Wells, Carmel, IN (US)

(21) Appl. No.: 18/381,694

(22) Filed: Oct. 18, 2023

Related U.S. Application Data
(60) Provisional application No. 63/417,919, filed on Oct. 20, 2022.

(51) Int. Cl. A61B 5/00; A61B 5/0205

(52) U.S. Cl. CPC A61B 5/4393; A61B 5/002; A61B 5/02055; A61B 5/4866; A61B 5/6831; A61B 5/7405; A61B 5/743; A61B 5/7475; A61B 2560/0214; A61B 2560/0443; A61B 2562/0219

(57) ABSTRACT

An electronic device called HELU (Hawaiian for tally) which can monitor medical body metrics and quantify/measure sexual performance as a number of strokes and pressure achieved as data for analyzing erectile dysfunction. The device can measure body metrics during sexual activity and at rest. The measurement data can be monitored, recorded and displayed, and analyzed/compared for a predetermined set of body metrics such as heart rate, pulse, blood pressure, calories burned, the tally and of strokes, and the pressure achieved during activity or at rest. The HELU communicates wirelessly to personal communication devices—smart phones, tablets, laptops, and personal computers. Analysis is supported by graphs displaying the data of body metrics and erectile dysfunction data. The data can be evaluated and considered for medical analysis, for physical status, for progress toward goals, and for games, competitive contests, and for personal amusement.

### CROSS-REFERENCE TO RELATED APPLICATIONS

This application claims the benefit of United States Provisional Patent Application with Ser. No. 63/417,919 filed Oct. 20, 2022, by James Spear and Josh Wells. The application is entitled “Special Performance . . . Analyzer (SPARTAN) Monitoring Device worn on a Penis during Sexual Activity”.

### FIELD OF INVENTION

This invention relates to an electronic monitoring device called “HELU” [Hawaiian for TALLY] which Measures Body Metrics like Pulse, Heart Rates, and Blood Pressure at rest, and which analyzes erectile performance that is used to monitor, analyze and optimize all characteristics of Sexual Activity and Erectile Dysfunction. The performance and analyzer (HELU) device is worn on the penis of the individual person during sexual activity or at rest. Called the HELU, the device relates to a kind of monitoring and medical devices, more particularly to a kind of electronic intelligence sexual function monitoring, diagnosing instrument. This relates to physiology detection apparatus fields to be used for male sex organ erection more particularly, to penis intelligence ring. The detection of function records the blood pressure, tumescence or rigidity and potentially other body metrics. The HELU relates to a device for measuring body metrics such as blood pressure, heart rate, pulse, and which analyzes erectile performance that is used to monitor, analyze and optimize all characteristics of Sexual Activity and Erectile Dysfunction. This is all based on wireless data transmission of the physiological changes at the penis during the performance of the sexual act. Baseline data is achieved by monitoring the data when the person is at rest.

### FEDERALLY SPONSORED RESEARCH

None.

### SEQUENCE LISTING OR PROGRAM

None.

### BACKGROUND-FIELD OF INVENTION and PRIOR ART

As far as known, there is no special electronic monitor for measuring body metrics and analyzing erectile dysfunction which is worn on a penis during sexual activity or the like. It is believed that this product is unique in its design and technologies.

### BACKGROUND

This background is for a special electronic monitor for measuring body metrics and analyzing erectile dysfunction which is worn on a penis during sexual activity or the like. The sensing and measurement of body metrics like heart rates, pulse, blood pressure, calories being burned has been in the medical field for many years, but not traditionally monitored during sexual activity. An objective device and manner to monitor and compare sexual arousal and analyze erectile performance in human beings has been attempted over the years by means which detect the swelling and color changes of parts of the male and female human body due to engorgement with blood in response to sexual arousal. Sexual performance and erectile dysfunction have not been as easy to track. The old adage—it is not the pen but penmanship—comes to mind. However, how can this be objectively tracked without emotion or the relationship of the parties biasing and affecting the results. Moods and the current emotion play a role, but is there a scientific manner to objectively measure one's sexual performance? Yes. Erectile performance can be analyzed and the pressure and force exerted during sexual activity is a common metric. It is believed that up to this present invention, pressure and force exerted have not been measured.

Measuring and monitoring rigidity and erection of a penis and arterial-venous flows has been attempted but no real medical device for measuring sexual function. The sensing and measurement of sexual arousal in human beings has been attempted over the years by means which detect the swelling and color changes of parts of the male and female human body due to engorgement with blood in response to sexual arousal. The prior apparatus and methods of determining male sexual arousal are primarily based on the enlargement in diameter of the penis in response to sexual arousal. Such changes in diameter are primarily measured using three types ox devices; a mercury-in-rubber detector, an iridium gallium detector, and a flexible metal detector. However, each of the three devices are resistant to size changes and thus require a certain amount of force to deform or stretch which can skew the actual measurements. Another known method of detecting spontaneous erections is to wrap a strip; of postage stamps about the penis to form a continuous ring before going to sleep. A positive indication is obtained if the ring of stamps bursts during sleep. This method relates only to the onset of tumescence and does not indicate whether the penis has become rigid. Therefore, it is desirable to provide a technique for detecting and accurately measuring both the degree of tumescence and the performance or stroke pressure achieved during the sexual act. Also, there are various sex products are available in the market. Some of them are beneficial, however, several, of them are merely a gimmick of one sort or another.

Pedometers are designed to detect vertical movement at the hip and so measure the number of steps and provide an estimate of distance walked. They cannot provide information on the temporal pattern of physical activity, or the time spent in different activities at different intensity. Graded exercise stress tests can identify hypertensive responses to activity and clinically significant electrocardiographic abnormalities during exercise and are thereby highly useful for providing a safe and effective cardiorespiratory exercise intensity range for at-risk patients. In healthy adults, heart rate is useful but not essential for the monitoring of exercise intensity. These sophisticated fitness trackers can be worn on the wrist, clipped to clothing, or incorporated into smartphones. Most of these trackers utilize an accelerometer to monitor movement, even during sleep, and some versions add GPS technology. Optical sensors for heart rate are common and provide useful data to optimize exercise intensity. Virtually all monitors are synchronized with smartphones or personal computers for tracking and graphic display of physical activity, calorie expenditure, and more. Modern pedometers work in a very similar way but are partly electronic. Open one up and one will find a metal pendulum (a hammer with a weight on one end) wired into an electronic counting circuit by a thin spring. Normally the circuit is open and no electric current flows through it. As a person takes a step, the hammer swings across and touches a metal contact in the center, completing the circuit and allowing current to flow. The flow of current energizes the circuit and adds one to his step count. As one completes the step, the hammer swings back again (helped by the spring) and the circuit is broken, effectively resetting the pedometer ready for the next step. More sophisticated pedometers (including some of the better ones made by Omron) work entirely electronically. Since they have no moving parts, tend to be longer-lasting, more reliable, and more accurate. They dispense with the swinging pendulum-hammer and measure the steps of the person with two or three accelerometers instead. These are microchips arranged at right angles that detect minute changes in force as one moves his/her legs. There are also digital devices that use more advanced programs to monitor and gauge a person's movement over time.

The Fitbit RTM is an always-on electronic pedometer, that in addition to counting steps also displays distance traveled, altitude climbed (via a number of flights of steps count), calories burned, current intensity, and time of day. Worn in an armband at night, this device also purports to measure the length and quality of a user's sleep. Inbuilt is a daily target, of 10,000 steps and 10 flights of stairs. Connected by USB with a computer, the user's data is automatically uploaded and displayed via a web-based profile page, which keeps track of historical data, to which can be added food consumption data. Fitbit's trackers have evolved in terms of functionality considerably since their introduction. Fitbit trackers are equipped with photoplethysmography sensors, which is the small green light one sees blinking on the tracker's underside. This light essentially monitors the volume of blood in one's wrist and uses that to calculate the number of heartbeats in a minute. When one is exercising, his heart rate increases, and the Fitbit tracker will tell him just how high or low it is. It also gives insights on how one can improve his/her performance and maintain his heart rate in a healthy range. Fitbit trackers can identify different types of exercises and place them in three different heart rate zones: Cardio, Peak, and Fat Burn zones. It uses a person's heart rate to determine which Zone he falls in and even sets challenges for him to focus on. This is great for people who want to improve their fitness and keeps them motivated. Certain Fitbit models are capable of tracking other health metrics too, such as oxygen saturation, breathing, a menstrual cycle, and even stress levels. Some Fitbit models also tell a person about his sleep quality when he wears them overnight and let him know when he is not sleeping enough. One of the newest Fitbit models, known as the Fitbit Luxe, offers more accurate fitness tracking, including swimming tracking. Fitbit also lets one track his daily meals and log calories.

A Fitbit uses a 3-axis accelerometer that can track the number of steps taken, in addition to a few other features such as distance traveled, calories burned, and number of floors climbed. An accelerometer is an electromechanical device that measures acceleration forces. When used in the Fitbit, the accelerometer senses movement, or vibrations. The more sensitive an accelerometer is, the more movement. To be:

able to count the number of steps there is a finely tuned, algorithm that detects unique motion patterns. The motion patterns must be large enough to meet the specific threshold of the algorithm, meaning that some steps could go uncounted if walking on very soft carpet or steps may be added if driving on a bumpy road and the algorithm's threshold is met. When one buys a Fitbit, they insert their information (weight, height, gender, etc.) onto the app/website, which allows for the program to generate data such as calories burned based off the steps traveled, type and duration of exercise, and the user's Personal information. An accelerometer is a tool that measures proper acceleration. Proper acceleration is the acceleration (the rate of change of velocity) of a body in its own instantaneous rest frame; this is different from coordinate acceleration, which is acceleration in a fixed coordinate system. For example, an accelerometer at rest on the surface of the Earth will measure an acceleration due to Earth's gravity, straight upwards (by definition) of g 9.81 m/s². By contrast, accelerometers in free fall (falling toward the center of the Earth at a rate of about 9.81 m/s²) will measure zero.

Accelerometers have many uses in industry and science. Highly sensitive accelerometers are used in inertial navigation systems for aircraft and missiles. Vibration in rotating machines is monitored by accelerometers. They are used in tablet computers and digital cameras so that images on screens are always displayed upright. In unmanned aerial vehicles, accelerometers help to stabilize flight. When two or more accelerometers are coordinated with one another, they can measure differences in proper acceleration, particularly gravity, over their separation in space—that is, the gradient of the gravitational field. Gravity gradiometry is useful because absolute gravity is a weak effect and depends on the local density of the Earth, which is quite variable. Single—and multi-axis accelerometers can detect both the magnitude and the direction of the proper acceleration, as a vector quantity, and can be used to sense orientation (because the direction of weight changes), coordinate acceleration, vibration, shock, and falling in a resistive medium (a case in which the proper acceleration changes, increasing from zero). Micromachined microelectromechanical systems (MEMS) accelerometers are increasingly present in portable electronic devices and video-game controllers, to detect changes in the positions of these devices. There are three different types of accelerometers, and they are each designed to efficiently function in their intended environments. The three types are: piezoelectric, piezoresistance and capacitive. A piezoelectric accelerometer utilizes the piezoelectric effect (piezoelectric materials produce electricity when put under physical stress) to sense change in acceleration. Piezoelectric accelerometers are most commonly used in vibration and shock measurement. Piezoresistance accelerometers are much less sensitive than piezoelectric accelerometers, and they are better suited to vehicle crash testing. A piezoresistance accelerometer increases its resistance in proportion to the amount of pressure applied to it. The third and most commonly used type of accelerometer is the capacitive accelerometer. Capacitive accelerometers use a change in electrical capacitance to determine an object's acceleration. When the sensor undergoes acceleration, the distance between its capacitor plates changes as the diaphragm of the sensor moves. Most accelerometers are miniscule, and they are often referred to as Micro-Electro-Mechanical Systems (MEMS) accelerometers. Because of their size and affordability, they are embedded in a myriad of hand-held electronic devices (such as phones, tablets, and video game controllers). In phones and tablets, the accelerometer is responsible for “flipping” the screen when the device is rotated. Accelerometers are also used by zoologists (to track the movement of animals in the wild), engineers (especially in collision experiments) and factories (to monitor the vibration of machinery).

Several smartwatches that measure BP (Blood Pressure) have recently been introduced. Most of these wearables measure BP through PTT (Pulse transit time) from a pulse wave measurement at the wrist. Several manufacturers have recently introduced medical tricorders that measure BP among other vital signs. The BodiMetrics Performance Monitor (BodiMetrics LLC, Manhattan Beach, California, United States) uses electrocardiogram and photoplethysmograph data to estimate BP through PTT. The Bodimetrics tricorder obtains measurements with a 20-second scan of the user's fingertip at heart level but only provides systolic BP data. Calibration to a validated cuff-type device is required prior to standalone use. Microelectromechanical systems (MEMS), also written as micro-electro-mechanical systems (or microelectronic and microelectromechanical systems) and the related micromechatronics and microsystems constitute the technology of microscopic devices, particularly those with moving parts. They merge at the nanoscale into nanoelectron mechanical systems (NEMS) and nanotechnology. MEMS are also referred to as micromachines in Japan and microsystem technology (MST) in Europe. MEMS are made up of components between 1 and 100 micrometers in size (i.e., 0.001 to 0.1 mm), and MEMS devices generally range in size from 20 micrometres to a millimetre (i.e., 0.02 to 1.0 mm), although components arranged in arrays (e.g., digital micromirror devices) can be more than 1000 mm². They usually consist of a central unit that processes data (an integrated circuit chip such as microprocessor) and several components that interact with the surroundings (such as microsensors). Because of the large surface area to volume ratio of MEMS, forces produced by ambient electromagnetism (e.g., electrostatic charges and magnetic moments), and fluid dynamics (e.g., surface tension and viscosity) are more important design considerations than with larger scale mechanical devices. MEMS technology is distinguished from molecular nanotechnology or molecular electronics in that the latter two must also consider surface chemistry.

### Problem Solved

The improvement and problem solved as to Special Performance and erectile performance Analyzer (HELU) Device worn on a Penis during Sexual Activity include: Is adjustable with a universal adjustable strap or made in predetermined sizes; Connects wirelessly with Bluetooth/wireless connection to cellular SMART phone, computer, tablet or electronic monitor—Apple and Android/PC compatible; Can be graphed or charted for comparison to previous self or other people's performances; Has multiple pocket/pouches for instrumentation and meters; Has a rechargeable power system/batteries or diodes; Notifies user and sexual partner of metrics; Can make/take multiple measurements of body metrics with sensors, pedometer like accelerometers, and Fit-Bit like electronic packages to measure metrics, medical monitoring instrumentation; Has sound and lights—continuous, strobe, and multi-colored; Is dishwasher safe, can be sterilized, and hot water washed; and Is waterproof and/or water resistant.

Some of the functions anticipated to be monitored improvement and problem solved as to Special Performance and erectile performance Analyzer (HELU) Device worn on a Penis during Sexual Activity can include: Measured and monitored-Erections frequency and duration/Erectile Dysfunction/ED indicators; PEDOMETER—strokes before ejaculation; Force and Pressures continuously monitored; maximum force achieved; HEART RATE; BLOOD PRESSURE; BLOOD OXYGEN; TEMPERATURE; STRESS MONITOR; and CALORIE COUNTER. Optional features—VIBRATOR; and/or OPTIONAL SCROTUM LOOP.

### PRIOR ART

It is believed that this product is unique in its design and technologies. A novelty search revealed:

A. Chinese Patent CN109124581A by China issued in 2018 for a Penis Intelligence Ring. This discloses penis intelligence rings, erection for Penis detects, including host and the socket ring being fixedly connected with host, ring is socketed as elastic material molding, host includes outer housing and set on power supply shell intracorporal control mainboard and connect with control mainboard, be integrated with Power Management Unit, MCU master controller and BLE Bluetooth module in control mainboard, MCU master controller respectively with Power Management Unit, BLE Bluetooth module circuit connection; Being socketed on ring has an open slot, one end of open slot is equipped with permanent magnet, the other end of open slot is equipped with Hall sensor, it Hall sensor and permanent magnet face and is electrically connected with control ma inboard, this program perceives the spacing amplitude of variation of open slot by changes of magnetic field that Hall sensor detests, to which record obtains the diameter change of penis, the maintenance duration of erection duration and parameters is recorded in conjunction with the fluctuating change of data, the program is implemented reliable, it is at low cost and operate conveniently, quickly monitoring tracking can be carried out comprehensively to user.

B. Chinese Patent CN109512387A by China issued in 2018 for an Electronic Intelligence Sexual Function, Monitoring, Diagnosing Instrument. This discloses kind of electronic intelligence sexual function monitoring, diagnosing instrument, including simulation stimulator, and simulation stimulator includes cylindric cavity, and cylindric cavity is axially reciprocal movement, and cylindric cavity inner wall adheres to air bag group, and air bag group surface is rough surface. It further include that temperature sensor, pressure sensor, displacement sensor, liquid sensor and whole control unit, temperature sensor, pressure sensor, displacement sensor, liquid sensor are all connected with control unit eventually, Temperature sensor is used to detect the gas-filling temperature of air bag group, and pressure sensor is used to detect the displacement of cylindric cavity axially reciprocating for detecting air bag group internal pressure, displacement sensor, and liquid sensor is located at the front of cylindric cavity. The present invention can monitor ejaculation latency time, bear stimulus intensity, motion profile and temporal correlation, every device of axial erection hardness and radial erection hardness.

C. Chinese Patent CN111214232A by China issued in 2020 for an Erection Function Detection Device and Erection Detection System Based on Wireless Transmission. This teaches an erection function detection device which comprises an annular body, wherein an air rag is formed in the annular body, an inflation inlet is formed in the annular body and used for inflating the air bag, and the elastic coefficient, of the annular body is gradually reduced from the inner side to the outer side; and a pressure sensor is arranged on the inner side of the annular body or in the air bag. The invention also discloses an erection function detection system based on wireless transmission, which comprises the erection function detection device. The invention judges the erection degree (hardness, length, circumference, erection duration, times, frequency and the like) by the pressure variation of the gas contained in the annular body caused by the volume change of the annular body, can realize real-time uninterrupted accurate detection, and does not bring discomfort.

D. Chinese Patent CN112702974A by China issued in 2021 for a Penile Ring Including Sensor. This demonstrates a penile ring adapted for occluding the urethra to prevent stress urinary incontinence is disclosed, having an annular body configured to encircle the penis, the annular body comprising an upper portion (10) including struts (20) extending in an arc around the upper portion and a lower portion (30) including one or more inflatable compression pads (40). The penile ring includes a sensor (110) to detect urine flow in the urethra and provide a signal to trigger the compression pad to expand inward to occlude to urethra, thereby preventing urine flow.

E. Chinese Patent CN204839454U by China issued in 2015 for An Intelligence Penis Ring. Thid shows an intelligence penis ring, including the ring body, be equipped with the detection device who is used for detecting male sex's sexual function on the ring body, detection device includes pressure sensor, temperature sensor, display screen, power module, storage module and central processing unit, pressure sensor, temperature sensor, display screen, storage module and power module all link to each other with central processing unit. The utility model discloses intelligence penis ring can realize the intellectual detection system assessment to male sex's sexual function, can diagnosis promptly and accurately treat impotence and premature ejaculation simultaneously, and it is very convenient to use.

F. Chinese Patent CN208610819U by China issued in 2015 for A Kind of Telotism Detection Device. This provides a kind of telotism detection device, including host and the falope ring being fixedly connected with the host, the host includes shell and the control mainboard being disposed in the housing, the falope ring is equipped with open slot, described open slot one end is equipped with permanent magnet, and the open slot other end is equipped with and the linear hall sensor for controlling mainboard electric connection. The utility model structure is simple, open slot is set in falope ring and linear hall sensor and permanent magnet are set in open slot two sides, thus the variation of penis diameter can be obtained according to the variation of open slot distance, manufacturing cost is low, and the detection device facilitates warring and uses.

G. Chinese Patent CN20922064OU by China issued in 2019 for a Kind of Medical Treatment Wearing Penis Ring Based on Smart Phone. Shown is a kind of a penis ring which is dressed in the medical treatment based on smart phone, is related to penis loop technique field. The utility model includes ring body, and ring body is made of silica gel material, and an open slot is offered in ring body, open slot both ends are fixed with control device by extension airbag, open slot both ends offer an arc-shaped slot, and the cross section of arc-shaped slot is circular configuration, and arc-shaped slot one end is fixed with a stop collar. The utility made is inflated by pressing air inlet capsule and by hose and air inlet pipe into connection capsule and extension airbag, extension airbag expansion squeezes shell and ring body, to improve the internal diameter of ring body, pass through rotary upper cover body, it can be vented when the gas vent in upper cover body and lower cover is opposite, sliding block is able to drive in arc-shaped slot by the elastic force of spring to slide, reduces the internal diameter of ring body, and solving the problems, such as that existing penis ring is not easy to adjust causes to be not suitable for penis personnel's wearing of different sizes.

H. Chinese Patent CN209733954U by China issued in 2019 as a Male Sexual Function Evaluation Instrument. The utility model discloses a male sexual function assessor, compare the apparatus at the hardness of time-recorder host computer including time-recorder host computer and setting, the hardness is compared the apparatus and is coupled with the time-recorder host computer, inputs the hardness testing result to the time-recorder host computer after comparing when surveying out the penis erection in the hardness, the time-recorder host computer begins and stops to count the back through received signal, matches the timing result and the hardness testing result and saves after. The utility model discloses a male sexual function assessor compares the setting of apparatus through time-recorder host computer and hardness, all right effectual realization penis hardness and time survey result pair the integration and store and read for the doctor can know male sexual function situation more comprehensively.

I. Korean patent KR200160104428A by Korea issued in 2016 for a Device for Sexual. Function. A sexual organ assisting device according to the present invention comprises a band unit and a sensor unit. Circles having spaces formed therein are formed on both ends of the band unit, and the circles are connected through a thin film to wrap the pens among sexual organs. The sensor unit is formed in the circles and senses a state of the penis. The sensor unit comprises a pulse oximetry device and a pressure measuring device. The pulse oximetry device measures the pulse or oxygen saturation of the penis, and the pressure measuring device the pressure of the penis when the penis is erect.

J. U.S. Pat. No. 4,469,108 by Goldstein was issued in 1984 for a Device for Measuring Penile Tumescence and Rigidity. It is a device for measuring tumescence and rigidity of the penis during erection has a first portion receivable about the base of the penis and actuable from an initial condition to an expanded condition by tumescence of the penis, and a second portion mountable to the penis at a location remote from the base for movement with the outer end of the penis. In a preferred embodiment, the first portion is constructed to restrict its return to the initial condition, and the first and second portions are connected to restrict movement of the second portion toward the first portion.

K. U.S. Pat. No. 4,911,176 by Timm et al. issued in 1990 for a Threshold Penile Rigidity Measuring Device. It is an apparatus for measuring predetermined penile rigidity or hardness in a penile tumescent event. The apparatus includes a band-like structure for releasably engaging a penis. Suitably attached and associated with band-like structure are releasable fasteners for detecting a predetermined force in a penile tumescent event whereby penile rigidity or hardness can be measured.

L. U.S. Pat. No. 6,050,959 by Card issued in 2000 for an Apparatus and Method. For measuring Sexual Arousal. This is an apparatus and method for sensing the swelling and shrinking of an elastic member based on movement and the angular changes to the surface of the elastic member between one or more portions of the surface thereof. The apparatus includes first and second parts which cooperate to produce an electrical signal indicative of the angular and distal positioning of the respective parts, with one of the parts preferably being a Hall effect device and the other a magnet. At least one part is mounted on a flexible base which attaches to a first portion of the surface of the elastic member for movement therewith with the other mounted for movement with a second surface portion of the elastic member. The apparatus and method are particularly adapted for use on a male penis and female breast for estimating sexual arousal based on the swelling of such body parts due to engorgement with blood which accompanies sexual arousal.

M. U.S. Pat. No. 9,247,904 by Hotaling issued in 2016 for an Apparatus for Noninvasively Measuring Corporal Hemodynamic and Mechanical Parameters for the Diagnosis of Sexual Function in Men and Women. This is a system and device for measuring sexual function includes an adjustable ring forming an opening to receive a penis, at least one diagnostic sensor positioned on an inner surface of the adjustable ring, wherein the ring automatically couples the diagnostic sensor to the penis throughout a range of ring diameters, and a microcontroller in communication with the at least one diagnostic sensor. At least one diagnostic sensor measures data during sexual activity and communicates the data to the microcontroller. In fact, in an example, the device is configured to be worn by a user during sexual activity.

N. U.S. Pat. No. 9,370,652 by Lin et al. issued in 2016 for a Desensitizing Device. This is a desensitizing device containing an electrical stimulation member optionally configured on a support member. The electrical stimulation member contains a control circuit, at least two electrodes, and an electricity supply element. The electricity supply element provides electricity to the control circuit, and the control circuit generates a stimulating current to the electrodes. By configuring a male genital with the support member so that the electrodes are in contact with the penis skin, the stimulating current produced by the control circuit is conducted to the electrodes and provides a low-strength subcutaneous nerve stimulation through the penis skin. The subcutaneous nerve is temporarily numbed and desensitized.

O. U.S. Pat. No. 11,389,369 by Bennett et al. issued in 2022 for an Apparatus and Method of Treating Premature Ejaculation. This is a neuromodulation such as ultrasound therapy having a cuff and transducer to removably position on a penis of a patient to treat premature ejaculation. The transducer stimulates a dorsal nerve of the penis to stimulate or inhibit the nerve pathway between the penis and the brain to delay ejaculation until the user or their partner desires to have an ejaculation occur.

P. US Patent application US 2005/0283044 by Chang issued in 2005 for a Flexible Ring for Penis. This is to provide a flexible ring comprising a cylindrical receptacle formed on an outer edge; a battery operated vibrator mounted in said receptacle; an annular flange around its inner edge; two opposite holes each through front and rear surfaces; wherein the ring is adapted to put on the base of an erect penis, the flange is adapted to tightly clamp the penis, and the holes are elastically deforming for increasing the resilience and the clamping force such that activating the vibrator will prolong the climax of sexual excitement by delaying ejaculation by the strong clamping force.

Q. US Patent application US 2009/0318755 by Adams et al. issued in 2009 for a Therapeutic Penis Ring. It shows a therapeutic vibrator that is a small barrel-shaped container housing a motor and at least one battery for the electric power source. The Therapeutic Vibrator is typically activated by an on/off switch or push-button at one end of the barrel, and when activated, the barrel will vibrate delivering massage to a specific body part. The barrel may comprise two halves that fasten together having recesses and cavities for battery (typically button-cell type), motor, switch wiring, etc. The barrel may then be contained within an appropriate vibrator body made from a material that is soft to the touch. The Therapeutic Vibrator may be used in a sex toy or in any other appropriate massaging device, such as small massagers for hands or faces.

R. US Patent application US 2010/0016759 by Lavoisier in 2010 for a Device and Method for Measuring and Treating.

It demonstrates the Rigidity And Erection. Of A Penis And Arterial-Venous Flows A measuring method enables the strength of the erector muscles (EM) causing the rigidity of the penis (P) of a patient and the arterial and venous variation ensuring the erection of the penis (P) of the patient to be measured and reinforced by placing a sensor (2, 50) around the penis (P) includes elements to receive the intracavernous pressure (ICP) and pressure variations of the penis (P), and at least one transducer (21, 55) integrated into or offset from the sensor (2, 50) allowing the pressure signal from the elements for receiving the pressure to be transformed into an electrical signal, and by connecting the sensor (2, 50) to a housing (35) including elements for amplifying and recording the signal from the transducer (21, 55), elements for analyzing the signal by a microcontroller allowing the fatigue state of the erector muscles (EM) to be measured and the variations in muscle strength of the erector muscles (EM) to be measured.

S. World publication WO 98/38914 by Erfinder et al. in 1998 for a Tumescence and Rigidity Measurement Device. It portrays a device for determining the tumescence and rigidity of a penis has a measurement value sensor (1) with an annular measurement loop (4) which can be stretched around the non-erect penis (3) and which can be stretched against a particular restoring force when the penis (3) swells, the measurement value sensor (1) emitting a signal representative of the loop extension. Known devices have a non-elastic tumescence measurement loop which is stretched with a predetermined force at predetermined intervals to determine the rigidity of the penis on the basis of its construction. In order to dispense with the complex control of two different measurement operations, the rigidity measurement value sensor (0) has a second measurement loop (5) which is arranged next to the first measurement loop (4) and can stretch against a restoring force high when the restoring force of the first measure ment loop (4).

T. World publication WO 01/12065 by Barnea in 2001 for a Penile Tumescence and Rigidity Monitoring Device. This shows a first embodiment of a device for measuring nocturnal penile tumescence and rigidity, two circular elastic sensors of different elasticity, placed around a penis, sense changes in penile circumference. A comparison of the instantaneous circumferences of the two sensors describes penile rigidity. Sensor circumference is measured by monitoring changes in electrical resistance across the sensor in a second embodiment, a single circular elastic sensor senses changes in penile circumference (describing tumescence), while rigidity is described by changes in amplitude of a plethysmography signal sensed by the single circular sensor. In a third embodiment, a photoplethysmographic sensor senses a penis plethysmography signal the signal means intensity describes penile tumescence, and changes in amplitude of the plethysmography signal describe changes in cardiac outlook.

U. World Publication WO 2017/035576 by Packham et al. in 2017 for an Adjustable Penile Compression Device. This is an adjustable compression device comprising an elongate rigid band provided to extend around a portion of the circumference of a penis. The band includes first and second side portions connected by an intermediate portion. An engagement member is secured between the first and second side portions such that the engagement member is rotatable about a rotational axis extending between the side portions. The engagement member is off-centered relative to said rotational axis such that an area defined between the engagement member and the band can be increased or decreased by rotation of the engagement member thereby adjusting pressure applied to the penis.

As can be observed, none of the prior art has anticipated or caused one skilled in the sexual performance measuring or monitoring devices and instrumentation to anticipate or see as obvious this invention by Spears and Wells—even to a person skilled in the ordinary art of the industry. The problems solved and the metrics monitored by the Special Performance and erectile performance Analyzer (HELU) Device worn on a Penis during Sexual Activity provide an answer to the long-sought question as to performance—is it the Pen, or the Penmanship! With the HELU device, sexual performance can now be objectively monitored.

### SUMMARY OF THE INVENTION

This is an electronic device called HELU (Hawaiian for tally) which can monitor medical body metrics and quantify/measure sexual performance as a force and pressure achieved. The device can measure body metrics during sexual activity and at rest. The measurement data can be monitored, recorded and displayed, and analyzed/compared for a predetermined set of body metrics such as heart rate, pulse, blood pressure, calories burned, the tally of strokes before ejaculation, and the pressure achieved during activity or at rest. The HELU communicates wirelessly to personal communication devices—smart phones, tablets, laptops, and personal computers. Analysis is supported by graphs displaying the data of body metrics, pressure, and number of strokes, and the like. The data can be evaluated and considered for medical analysis, for physical status, for progress toward goals, and for games, competitive contests, and for personal amusement.

The preferred embodiment of a Special Performance and erectile performance Analyzer (HELU) Device worn on a Penis during Sexual Activity comprised of: (a) a multi-pouch HELU device (2,3,4 pouches); (b) a means for removable securing the device to a penis, such means as a securing strap or closure band or alternatively a fixed device with a predetermined inside diameter and surface and outside diameter and outer surface; (c) at least one control and measurement component such as an accelerometer, sensors, pedometer like instrument Fit-Bit RTM electronics or the like; (d) a means of power battery, capacitor, LI-ION, NI-CD, alkaline, nickel metal hydride (NIMH), cobalt, rare earth and composite materials etc.—standard one use and rechargeable and a recharging plug/connection/recharge port; (e) a control means push button, switch; (f) at least one capillary and/or sensing lead from the penis to the control and measurement component; (g) a display of stokes number and force/pressure, pulse, blood pressure, calories burned, on the device; (h) a wireless connection/blue tooth; and (i) a set of options such as a light, LED, sound emitter wherein the HELU communicates with the wireless connection/blue tooth to a set of personal communication devices—such as smart phones, tablets, laptops, personal computers, and the like and then a multitude of potential graph results of body metrics, pressure and the like can be evaluated and considered for medical analysis and physical status of the individual, for progress toward goals, for games or competitive contests, or merely for novelty and personal amusement.

The newly invented Special Performance and erectile performance Analyzer (HELU) Device worn on a Penis during Sexual Activity can be manufactured at low volumes by very simple means and in high volume production by more complex and controlled systems.

### Objects and Advantages

There are several objects and advantages of the Special Performance and erectile performance Analyzer (HELU) Device worn on a Penis during Sexual Activity like Pulse, Heart Rates, and Blood Pressure at rest, and which Measures Performance as a Number of strokes and Pressure/Force of strokes during Sexual Activity. There are currently no known devices such as this that are effective at providing the objects of this invention. The HELU) device worn on a Penis during Sexual Activity for monitoring performance and measuring body metrics called a HELU—has various advantages and benefits:

| Item | Advantages |
|---|---|
| 1 | Is adjustable with a universal adjustable strap or |
| | made in predetermined sizes |
| 2 | Connects wirelessly with Bluetooth/wireless |
| | connection to cellular SMART phone, computer, |
| | tablet, or electronic monitor - Apple and |
| | Android/PC compatible |
| 3 | Can be graphed or charted for comparison to |
| | previous self or other people's performances |
| 4 | Has multiple pocket/pouches for instrumentation |
| | and meters |
| 5 | Has a rechargeable power system/batteries or |
| | diodes |
| 6 | Notifies user and sexual partner of metrics |
| 7 | Can make/take multiple measurements of body |
| | metrics with sensors, pedometer-like |
| | accelerometers, and Fit-Bit like electronic |
| | packages to measure metrics, medical monitoring |
| | instrumentation |
| 8 | Has sound and lights - continuous, strobe, and |
| | multi-colored |
| 9 | Is dishwasher safe, can be sterilized, and hot |
| | water washed. |
| 10 | Is waterproof and/or water resistant |

Finally, other advantages and additional features of the present Special Performance and erectile performance Analyzer (HELU) Device worn on a Penis during Sexual Activity will be more apparent from the accompanying drawings and from the full description of the device. For one skilled in the art of monitoring body metrics and/or sexual performance measuring and monitoring machines and instrumentation, it is readily understood that the features shown in the examples with this product are readily adapted to other types of sexual performance and body metric devices.

### DESCRIPTION OF THE DRAWINGS—FIGURES

The accompanying drawings, which are incorporated in and constitute a part of this specification, illustrate an embodiment of the Special Electronic Monitoring Device called HELU which Measures Body Metrics like Pulse, Heart Rates, and Blood Pressure at rest, and which Measures Performance as a Number and Pressure of strokes during Sexual Activity that is preferred. This is called a “HELU” since the word HELU is Hawaiian for tally or count. The drawings together with the summary description given above and a detailed description given below serve to explain the principles of the HELU device called a HELU. It is understood, however, that this device is not limited to only the precise arrangements and instrumentalities shown.

FIGS. 1A through 1D are sketches of options for HELU devices for measurement of sexual performance.

FIGS. 2A through 2D are sketches of the general HELU device from a side, top and perspective views with components and features noted.

FIGS. 3A through 3E are sketches add additional options of the HELU devices with the components and features shown from generally a top view.

FIGS. 4A through 4D are sketches of a HELU device from different views with features detailed.

FIGS. 5A through 5E are sketches of a HELU device with features provided.

FIGS. 6A through 6C are sketches of a prototype HELU device.

FIGS. 7A and 7B are sketches of the HELU device in use.

FIGS. 8A through 8E are sketches of an alternative embodiment of a body metrics, sexual performance, and erectile performance analyzing device with features provided.

FIGS. 9A through 9F are sketches of prior art and sexual performance and measurement devices.

### DESCRIPTION OF THE DRAWINGS—REFERENCE NUMERALS

The following list refers to the drawings:

TABLE B
Reference numbers

| Ref # | Description |
|---|---|
| 30 | body metrics, sexual performance and erectile |
| | performance analyzing device 30 for measurement |
| | monitoring, and testing metrics of an individual |
| 31 | concept 31 for HELU 30 |
| 32 | alternative embodiment 32 of a body metrics, |
| | sexual performance, and erectile performance |
| | analyzing device 30 for measurement, monitoring, |
| | and recording body metrics such as blood |
| | pressure, calories burned, heart rate, number of |
| | strokes and pressure at ejaculation, pulse |
| 35 | prototype HELU device 35 |
| 39 | multi-pouch HELU device (2, 3, 4 pouches 39-2, 39- |
| | 3, 39-4) |
| 40 | means 40 for removable securing the device 30 |
| 41 | securing strap 41 or closure band or |
| | alternatively a fixed device 30 with a |
| | predetermined inside diameter 66 and surface 72 |
| | and outside diameter 64 and outer surface 71 |
| 41A | adjustable end 41A with a plurality of apertures 43 |
| 41B | fixed end 41B with prong 42 |
| 42 | prong, stud, extension stud 42 |
| 43 | aperture, hole, punch openings 43 |
| 44 | retention loop 44 inter-connect loop 44 on |
| | aperture end 41A |
| 45 | display 45 of stroke number and pressure, pulse, |
| | blood pressure, calories burned, etc. |
| 46 | expanded portion 46 for controls and display 45 |
| 48 | secondary readout display 48 for body metrics and |
| | details for an erectile performance analyzer |
| 49 | slot, opening, access 49 to control component |
| | 70, 60 |
| 50 | control pouch, cavity, pocket 50 |
| 53 | external surface 53 for control |
| 55 | secondary pouches for batteries, controls, |
| | additional sensors, instruments |
| 57 | canal/channel/pathway 57 between pouches 50, 55 |
| 60 | secondary controls and measurement instruments 60 |
| | (accelerometers, sensors, counters, analyzer, |
| | counters, etc.) |
| 62 | encasement 62 for controls, instrument, |
| | batteries, containers, capsule |
| 64 | outside diameter 64 |
| 66 | inside diameter and encircling area 66 for penis |
| | 110 |
| 67 | light, LED, sound emitter, 67 |
| 68 | control means 68 push buttons, switch etc. |
| 70 | a control and measurement component 70 such as an |
| | accelerometer, sensors, pedometer like instrument |
| | Fit-Bit RTM electronics or the like |
| 71 | external surface 71 of HELU 30 |
| 72 | interior ring surface 72 of HELU 30 |
| 72A | sensor cover 72A |
| 73 | set of form/mold breaks 73 on interior of HELU 30 |
| 74 | rib 74 on interior ring of device |
| 75 | means of power 75 battery, capacitor, LI-ION, NI- |
| | CD, alkaline, nickel metal hydride (NIMH), |
| | cobalt, rare earth, and composite materials etc. - |
| | standard one use and rechargeable |
| 76 | recharging plug/connection/recharge port 76 |
| 77 | wireless connection/blue tooth 77 |
| 78 | optional scrotum loop 78 |
| 79 | interior ring 79 of prototype device 30, 35 |
| 80 | capillary and or sensing leads 80 |
| 81 | alternative means 81 for removable securing the |
| | alternative embodiment 32 of a body metrics, |
| | sexual performance, and stoke number and pressure |
| | for erectile performance analysis device 30 for |
| | measurement, monitoring, and recording body |
| | metrics |
| 82 | adjustable end 82 with a plurality of bead-like spheres 83 |
| 83 | bead-like spheres 83 linearly inter-connected by |
| | a cord/molded cord, string, chain |
| 84 | fixed end 84 with aperture 85 |
| 85 | aperture, hole, punch, openings 85 |
| 86 | holding extension 86 for adjusting length/ |
| | circumference of device 30 |
| 87 | cord/molded cord, string, chain 87 |
| 88 | end 88 of cord/string 87 for alternative means |
| | 81 for removable securing the alternative |
| | embodiment 32 of a body metrics, sexual |
| | performance, and stoke number and pressure for |
| | erectile performance analysis device 30 for |
| | measurement, monitoring, and recording body |
| | metrics |
| 100 | person 100 for measurement |
| 106 | personal communication devices 106 - such as |
| | smart phones, tablets, laptops, personal |
| | computers, and the like |
| 108 | graph results 108 of body metrics, pressure and |
| | number of strokes, and the like |
| 110 | penis 110 to be monitored |
| 500 | Prior Art 500 US 2005/0283044 - Chang - 2005 - |
| | Flexible Ring for Penis |
| 510 | Prior Art 510 KR20160104428A Korean - 2016 Device |
| | for Sexual Function |
| 520 | Prior Art 520 U.S. Pat. No. 11,389,369 Bennett et al. - 2022 |
| | Apparatus and Method of Treating Premature |
| | Ejaculation |
| 530 | Prior Art 530 US 2009/0318755 Adams et al. in |
| | 2009 Therapeutic Penis Ring |
| 540 | Prior Art 540 US 2010/0016759 Lavoisier - 2010 |
| | Device and Method for Measuring and Treating the |
| | Rigidity and Erection |
| | Of a Penis & Arterial-Venous Flows |
| 550 | Prior Art 550 WO 98/38914 Erfinder et al. - 1998 |
| | Tumescence and Rigidity Measurement Device |

### DETAILED DESCRIPTION OF PREFERRED EMBODIMENT

This invention relates to a Special Performance and erectile performance Analyzer (HELU) Device worn on a Penis during Sexual Activity which Measures Body Metrics like Pulse, Heart Rates, and Blood Pressure at rest, and which helps analyze Erectile Performance during Sexual Activity. The HELU device relates to a kind of monitoring and medical devices, more particularly to a kind of electronic intelligence for body metrics such as heart rate, blood pressure, calorie burn, pulse, etc. and for objectively measuring sexual function monitoring, diagnosing instrument. This relates to physiology detection apparatus fields to be used for male sex organ erection more particularly, to penis intelligence ring. The detection of function records the blood pressure, tumescence or rigidity and potentially other body metrics. The HELU relates to a sexual function detection device, particularly for an erection function detection device the number of strokes before ejaculation and the pressure achieved or force of the strokes—all based on wireless transmission of the physiological changes at the penis during the performance of the sexual act.

The advantages for the body metrics, sexual performance and erectile performance analyzing device 30 for measurement monitoring, and testing metrics of an individual during Sexual Activity. Succinctly the benefits are that the device:
A. Is adjustable with a universal adjustable strap or made in predetermined sizes;
B. Connects wirelessly with Bluetooth/wireless connection to cellular SMART phone, computer, tablet, or electronic monitor—Apple and Android/PC compatible;
C. Can be graphed or charted for comparison to previous self or other people's performances;
D. Has multiple pocket/pouches for instrumentation and meters;
E. Has a rechargeable power system/batteries or diodes;
F. Notifies user and sexual partner of metrics;
G. Can make/take multiple measurements of body metrics with sensors, pedometer like accelerometers, and Fit-Bit like electronic packages to measure metrics, medical monitoring instrumentation;
H. Has sound and lights—continuous, strobe, and multi colored;
I. Is dishwasher safe, can be sterilized, and hot water washed; and
J. Is waterproof and/or water resistant.

The preferred embodiment of the Electronic Monitoring Device called HELU which Measures Body Metrics like Pulse, Heart Rates, and Blood Pressure at Rest, and which creates a well-defined performance measurement that is used to monitor, analyze and optimize all characteristics of Sexual Activity and Erectile Dysfunction worn on a Penis during Sexual Activity comprised of: (a) a multi-pouch HELU device (2,3,4 pouches 39-2, 39-3, 39-4); (b) a means 40 for removable securing the device 30 to a penis, such means as a securing strap 41 or closure band or alternatively a fixed device 30 with a predetermined inside diameter 66 and surface 72 and outside diameter 64 and outer surface 71; (c) at least one control and measurement component 70 such as an accelerometer, sensors, pedometer like instrument Fit-Bit RTM electronics or the like; (d) a means of power 75 battery, capacitor, LI-ION, NI-CD, alkaline, nickel metal hydride (NIMH), cobalt, rare earth and composite materials etc.—standard one use and rechargeable and a recharging plug/connection/recharge port 76; (e) a control means 68 push button, switch; (f) at least one capillary and/or sensing lead 80 from the penis to the control and measurement component 70; (g) a display 45 of stroke number and pressure, pulse, blood pressure, calories burned, on the device 30; (h) a wireless connection/blue tooth 77; and (i) a set of options such as a light, LED, sound emitter, 67 wherein the HELU communicates with the wireless connection/blue tooth 77 to a set of personal communication devices 106—such as smart phones, tablets, laptops, personal computers, and the like and then a multitude of graph results 108 of body metrics, pressure, and number of strokes can be evaluated and considered for medical analysis and physical status of the individual, for progress toward goals, for games or competitive contests, or merely for novelty and personal amusement.

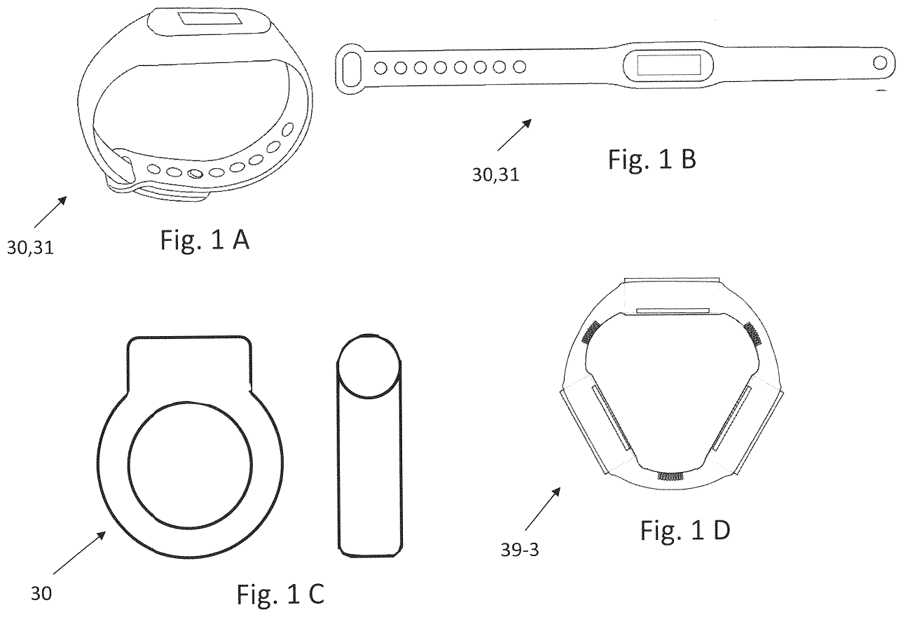

There is shown in FIGS. 1-9 a complete description and operative embodiment of the Special Electronic Monitoring Device 30 called HELU which Measures Body Metrics like Pulse, Heart Rates, and Blood Pressure at rest, and which creates a well-defined performance measurement that is used to monitor, analyze, and optimize all characteristics of Sexual Activity and Erectile Dysfunction. In the drawings and illustrations, one notes well that FIGS. 1-9 demonstrate the general configuration and use of this product. The various example uses are in the operation and use section, below.

The accompanying drawings, which are incorporated in and constitute a part of this specification, illustrate an embodiment of the Special Electronic Monitoring Device 30 called HELU which Measures Body Metrics like Pulse, Heart Rates, and Blood Pressure at rest, and which creates a well-defined performance measurement that is used to monitor, analyze, and optimize all characteristics of Sexual Activity and Erectile Dysfunction that is preferred. The drawings together with the summary description given above and a detailed description given below serve to explain the principles of the electronic device (HELU) Device 30. It is understood, however, that the HELU device 30 is not limited to only the precise arrangements and instrumentalities shown. Other examples of sexual performance and body metric measuring devices and instrumentation devices and their uses are still understood by one skilled in the art of measuring body functions and metrics to be within the scope and spirit shown here.

FIGS. 1A through 1D are sketches of options for electronic device 30 called a HELU for measurement of sexual performance and body metrics. Shown here are: a body metrics, sexual performance, and erectile performance analyzing device 30 for measurement monitoring, and testing metrics of an individual; a concept 31 for HELU 30; a prototype HELU device 35; and a multi-pouch HELU device (2,3,4 pouches 39-2, 39-3, 39-4).

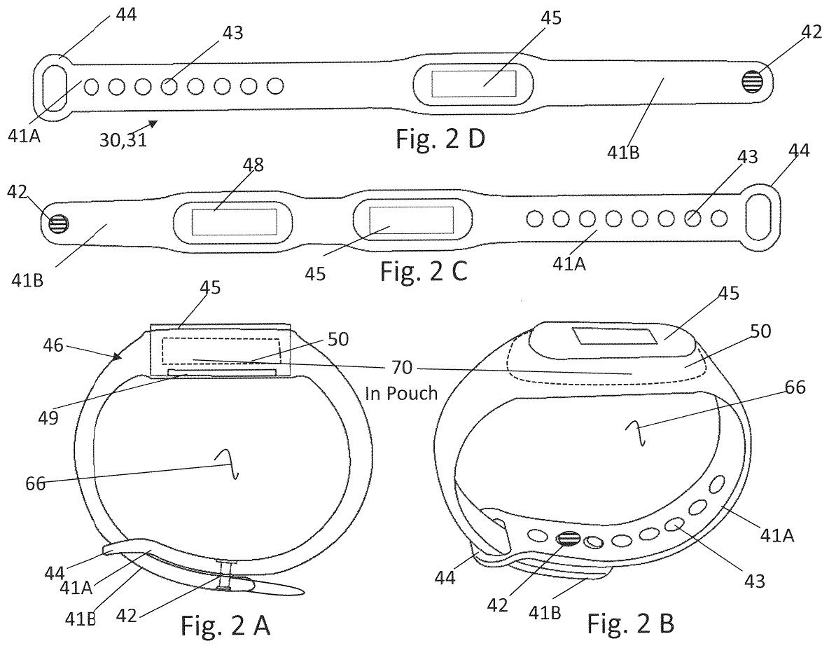

FIGS. 2A through 2D are sketches of the general HELU device 30 from a side, top and perspective views with components and features noted. Demonstrated here are the following features and components: a body metrics, sexual performance and erectile performance analyzing device 30 for measurement monitoring, and testing metrics of an individual; a concept 31 for HELU 30; a means 40 for removable securing the device 30; an securing strap 41 or closure band or alternatively a fixed device 30 with a predetermined inside diameter 66 and surface 72 and outside diameter 64 and outer surface 71; an adjustable strap 41A with a plurality of apertures 43; a fixed end 41B with prong 42; a prong, stud, extension stud 42; an aperture, hole, punch openings 43; a retention loop 44 inter-connect loop 44 on aperture end 41A; a display 45 of stroke number and pressure, pulse, blood pressure, calories burned; an expanded portion 46 for controls and display 45; a secondary readout display 48 for body metrics and details for an erectile performance analyzer; a slot, opening, access 49 to control component 70,60; and a control pouch, cavity, pocket 50.

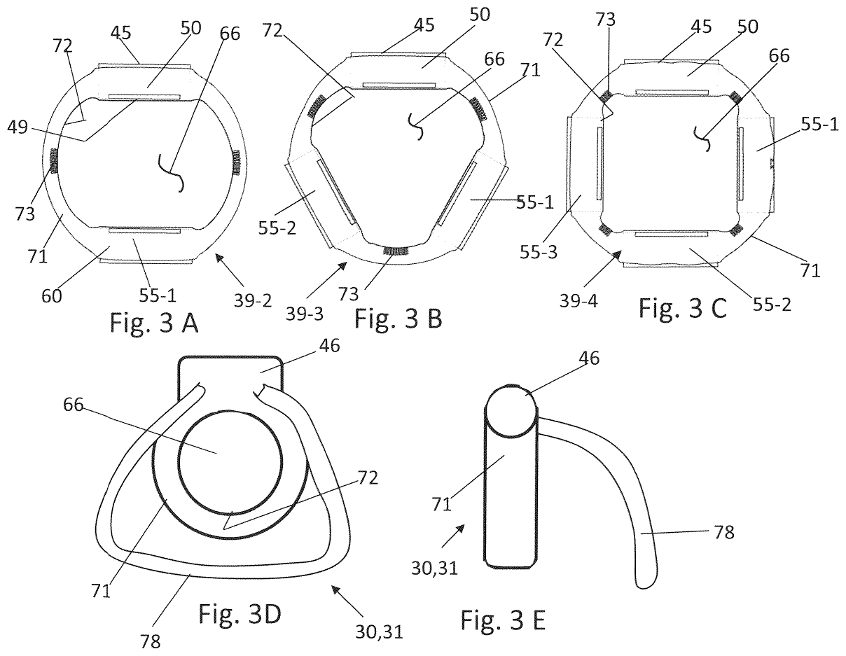

FIGS. 3A through 3E are sketches of additional options of the HELU devices 30 with the components and features shown from generally a top view. Portrayed here are: a body metrics, sexual performance and erectile performance analyzing device 30 for measurement monitoring, and testing metrics of an individual; a concept 31 for HELU 30; a multi-pouch HELU device (2,3,4 pouches 39-2, 39-3, 39-4); a predetermined inside diameter 66 and surface 72 and outside diameter 64 and outer surface 71; a display 45 of stroke number and pressure, pulse, blood pressure, calories burned, etc.; a secondary readout display 48 for body metrics and details for an erectile performance analyzer; a slot, opening, access 49 to control component 70,60; a control pouch, cavity, pocket 50; a secondary controls and measurement instruments 60 (accel-erometers, sensors, counters, analyzer, counters, etc.); an encasement 62 for controls, instrument, batteries, containers, capsule; an outside diameter 64; an inside diameter and encircling area 66 for penis 110; a control and measurement component 70 such as an accelerometer, sensors, pedometer like instrument Fit-Bit RTM electronics or the like; an external surface 71 of HELU 30; an interior ring surface 72 of HELU 30; and a set of form/mold breaks 73 on interior of HELU 30.

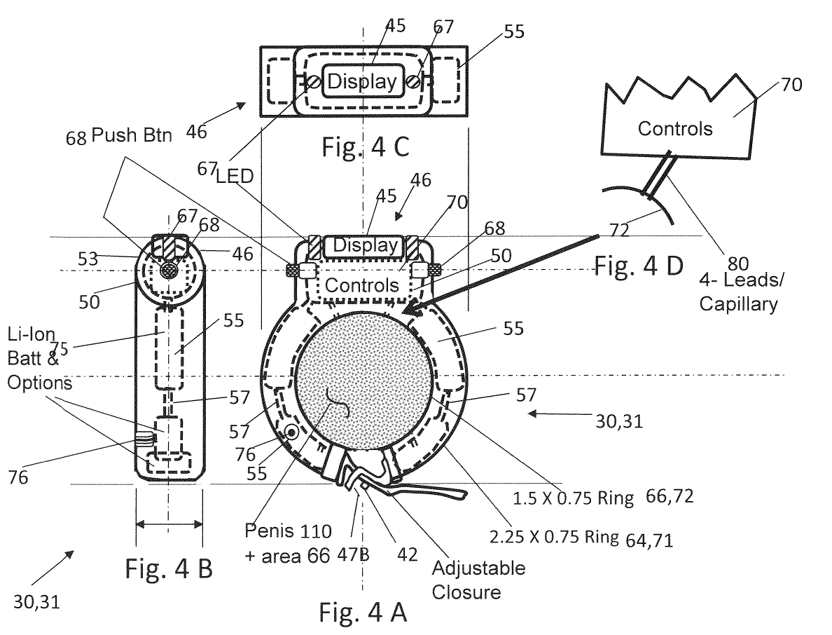

FIGS. 4A through 4D are sketches of another embodiment of the HELU device 30 from different views with features detailed. Viewed in these sketches are: a body metrics, sexual performance and erectile performance analyzing device 30 for measurement monitoring, and testing metrics of an individual; a concept 31 for HELU 30; a means 40 for removable securing the device 30; a securing strap 41 or closure band or alternatively a fixed device 30 with a predetermined inside diameter 66 and surface 72 and outside diameter 64 and outer surface 71; an adjustable strap 41A with a plurality of apertures 43; a fixed end 41B with prong 42; a prong, stud, extension stud 42; an aperture, hole, punch openings 43; a retention loop 44 inter-connect loop 44 on aperture end 41A; a display 45 of stroke number and pressure, pulse, blood pressure, calories burned, etc.; an expanded portion 46 for controls and display 45; a secondary readout display 48 for body metrics and details for an erectile performance analyzer; a slot, opening, access 49 to control component 70,60; a control pouch, cavity, pocket 50; an external surface 53 for control; a secondary pouches for batteries, controls, additional sensors, instruments; a series of canal/channel/pathway 57 between pouches 50, 55; a secondary controls and measurement instruments 60 (accelerometers, sensors, counters, analyzer, counters, etc.); an encasement 62 for controls, instrument, batteries, containers, capsule; an outside diameter 64; an inside diameter and encircling area 66 for penis 110; a light, LED, sound emitter, 67; a control means 68 push button, switch etc.; a control and measurement component 70 such as an accelerometer, sensors, pedometer like instrument Fit-Bit RTM electronics or the like; an external surface 71 of HELU 30; an interior ring surface 72 of HELU 30; a means of power 75 battery, capacitor, LI-ION, NI-CD, alkaline, nickel metal hydride (NIMH), cobalt, rare earth and composite materials etc.—standard one use and rechargeable; a recharging plug/connection/recharge port 76; and a capillary and or sensing leads 80; a person 100 for measurement.

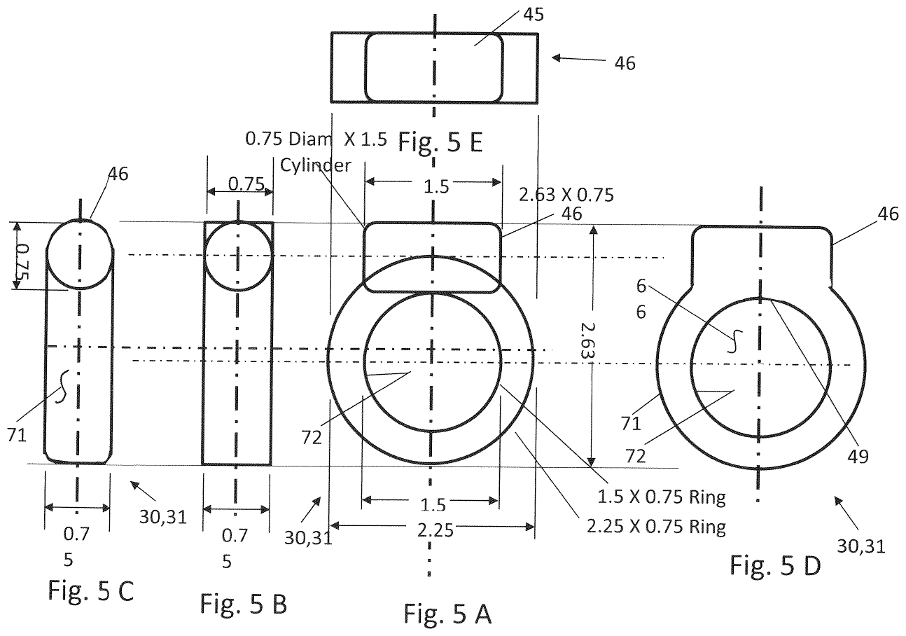

FIGS. 5A through 5E are sketches and one example of measurements for the alternative HELU device 30 with features noted. Demonstrated sizing and configurations are as follows: a body metrics, sexual performance and erectile performance analyzing device 30 for measurement monitoring, and testing metrics of an individual; a concept 31 for HELU 30; a fixed device 30 with a predetermined inside diameter 66 and surface 72 and outside diameter 64 and outer surface 71; a display 45 of stroke number and pressure, pulse, blood pressure, calories burned, etc.; an expanded portion 46 for controls and display 45; a slot, opening, access 49 to control component 70,60; and an inside diameter and encircling area 66 for penis 110.

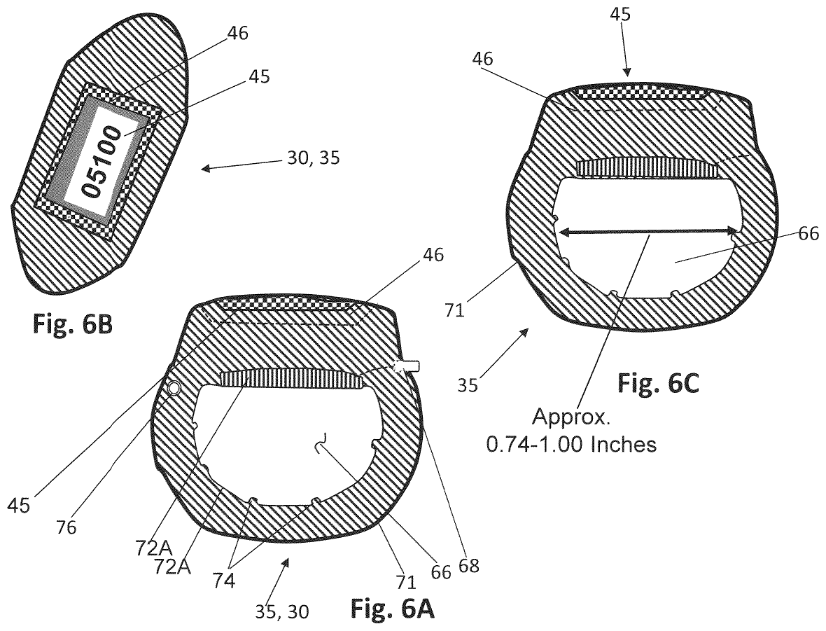

FIGS. 6A through 6C are sketches of a prototype HELU device 30. Shown are: a body metrics, sexual performance and erectile performance analyzing device 30 for measurement monitoring, and testing metrics of an individual; a prototype HELU device 35; a display 45 of stroke number and pressure, pulse, blood pressure, calories burned, etc.; an expanded portion 46 for controls and display 45; an inside diameter and encircling area 66 for penis 110; an external surface 71 of HELU 30; an interior ring surface 72 of HELU 30; an interior ring 79 of prototype device 30, 35; an interior ring surface 72 of HELU 30; a sensor cover 72A; and at least one rib(s) 74 on interior ring of device.

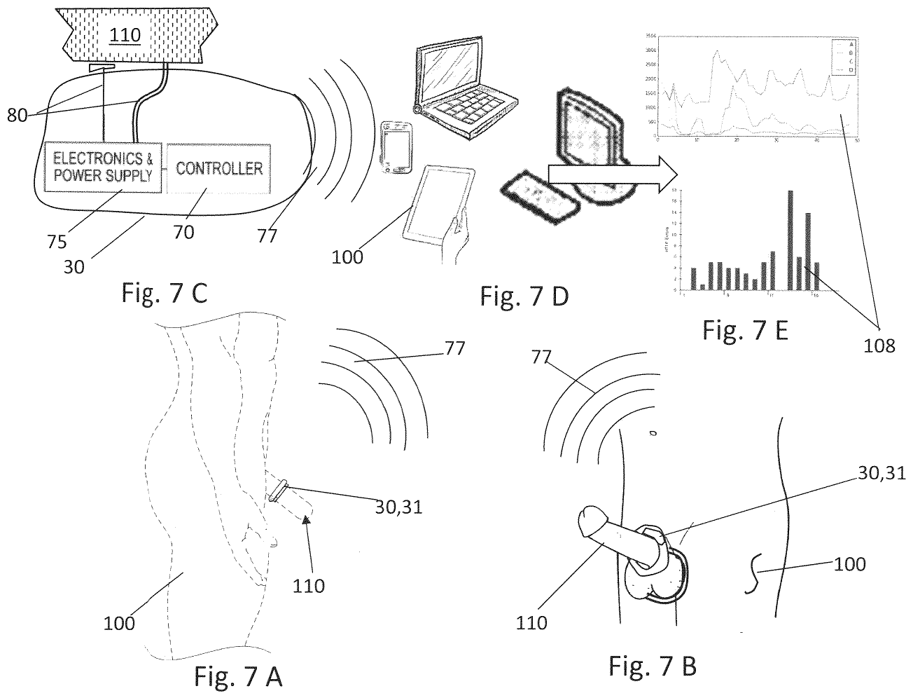

FIGS. 7A and 7B are sketches of the HELU device 30 in use. These are described in the Operation Section below.

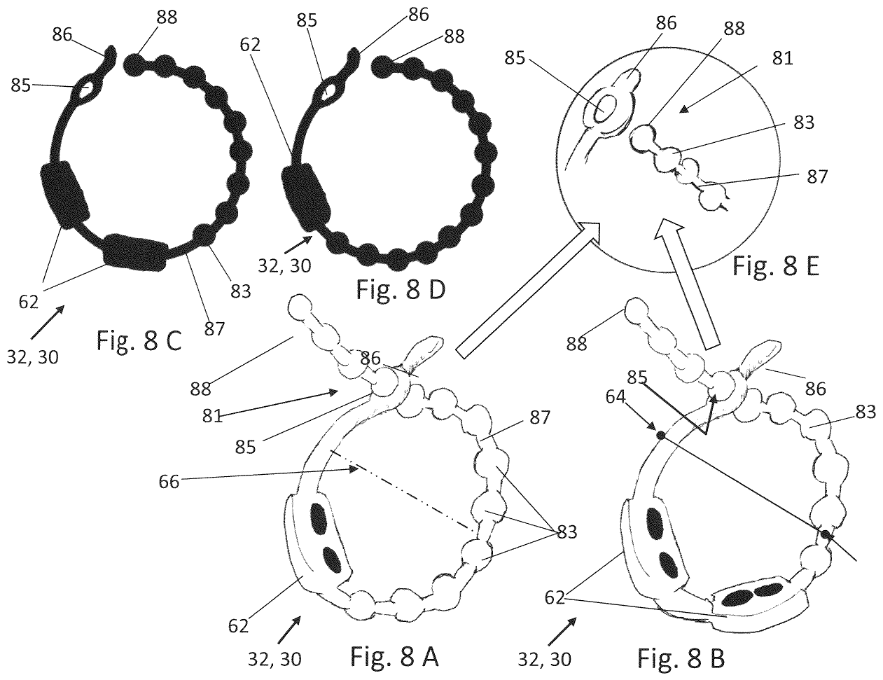

FIGS. 8A through 8E are sketches of an alternative embodiment 32 of a body metrics, sexual performance, and erectile performance analyzing device 30. The features and sketches include: an alternative embodiment 32 of a body metrics, sexual performance, and erectile performance analyzing device 30 for measurement, monitoring, and recording body metrics such as blood pressure, calories burned, heart rate, number of strokes and pressure at ejaculation, pulse; an alternative means 81 for removable securing the alternative embodiment 32 of a body metrics, sexual performance, and stoke number and pressure for erectile performance analysis device 30 for measurement, monitoring, and recording body metrics; an adjustable end 82 with a plurality of bead-like spheres 83 linearly inter-connected by a cord/molded cord, string, chain; a fixed end 84 with aperture 85 aperture, hole, punch, openings 85; a holding extension 86 for adjusting length/circumference of device 30; a cord/molded cord, string, chain 87; and an end 88 of cord/string 87 for alternative means 81 for removable securing the alternative embodiment 32 of a body metrics, sexual performance, and stoke number and pressure for erectile performance analysis device 30 for measurement, monitoring, and recording body metrics. The manner to operate the alternative embodiment is described in the Operations Section, below.

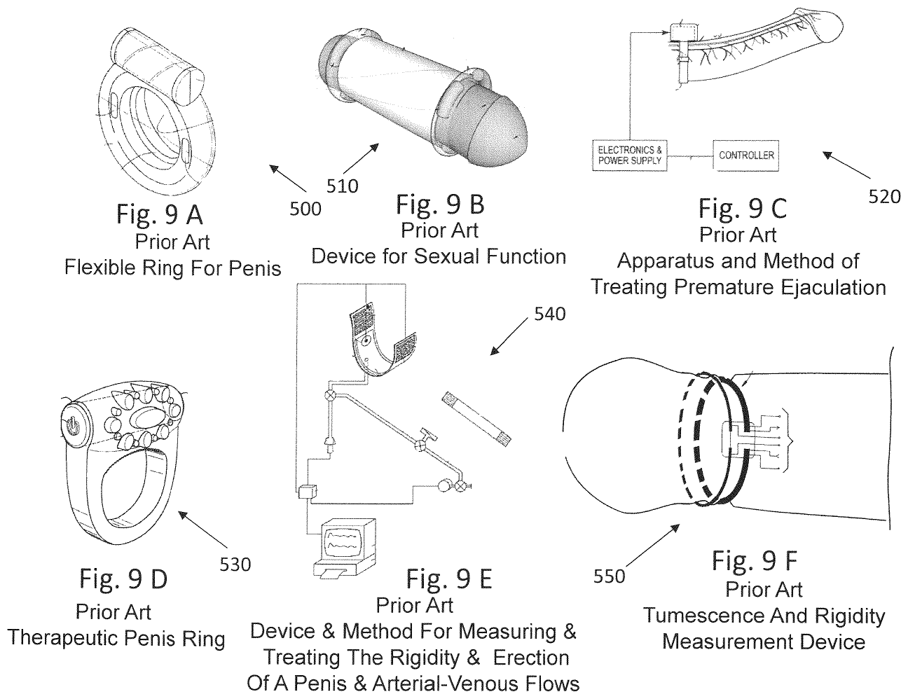

FIGS. 9A through 9F are sketches of prior art and sexual performance and measurement devices. Here former patents and applications for sexual monitoring and measurement devices for function of a male sexual organ and resulting performance. These include: Prior Art 500 US 2005/0283044-Chang—2005—Flexible Ring for Penis; Prior Art 510 KR20160104428A Korean—2016 Device for Sexual Function; Prior Art 520 U.S. Pat. No. 11,389,369 Bennett et al.—2022 Apparatus and Method of Treating Premature Ejaculation; Prior Art 530 US 2009/0318755 Adams et al. in 2009 Therapeutic Penis Ring; Prior Art 540 US 2010/0016759 Lavoisier—2010 Device and Method for Measuring and Treating the Rigidity and Erection. Of a Penis & Arterial-Venous Flows; and Prior Art 550 WO 98/38914 Erfinder et al.—1998 Tumescence and Rigidity Measurement Device. As can be seen, the Special Electronic Monitoring Device 30 called HELU which Measures Body Metrics like Pulse, Heart Rates, and Blood Pressure at rest, and which creates a well-defined performance measurement that is used to monitor, analyze and optimize all characteristics of Sexual Activity and Erectile Dysfunction worn on a Penis during Sexual Activity is a unique combination and use as described herein.

There are various materials and methods to produce a HELU device 30. The main enclosure and body of the HELU device 30 anticipates silicon, rubber, urethane, composite materials, metal—stainless steel, silver, gold, and platinum, wood, leather, silk, and synthetic fabrics. The means of power 75 battery, capacitor, LI-ION, NI-CD, alkaline, nickel metal hydride (NIMH), cobalt, rare earth, and composite materials etc.—standard one use and rechargeable.

The details mentioned here are exemplary and not limiting. Other types of specific components and manners specific to describing a Special body metrics, sexual performance and erectile performance analyzing device 30 called a HELU, can be added as a person having ordinary skill in this field (i.e. the field of the art of measuring body metrics and/or objectively measuring sexual performance with instrumentation and measuring devices).

### Operation of the Preferred Embodiment

The Special Electronic Monitoring Device 30 called HELU which Measures Body Metrics like Pulse, Heart Rates, and Blood Pressure at rest, and which creates a well-defined performance measurement that is used to monitor, analyze and optimize all characteristics of Sexual Activity and Erectile Dysfunction, has been described in the above embodiment. The manner of how the device operates is described below. One notes well that the description above and the operation described here must be taken together to fully illustrate the concept of the Special device 30 called a HELU. The preferred embodiment is a Special Electronic Monitoring Device 30 called HELU which Measures Body Metrics like Pulse, Heart Rates, and Blood Pressure at rest, and which creates a well-defined performance measurement that is used to monitor, analyze and optimize all characteristics of Sexual Activity and Erectile Dysfunction is worn on a Penis during Sexual Activity comprised of: (a) a multi-pouch HELU device (2,3,4 pouches 39-2, 39-3, 39-4); (b) a means 40 for removable securing the device 30 to a penis, such means as a securing strap 41 or closure band or alternatively a fixed device 30 with a predetermined inside diameter 66 and surface 72 and outside diameter 64 and outer surface 71; (c) at least one control and measurement component 70 such as an accelerometer, sensors, pedometer like instrument Fit-Bit RTM electronics or the like; (d) a means of power 75 battery, capacitor, LI-ION, NI-CD, alkaline, nickel metal hydride (NIMH), cobalt, rare earth and composite materials etc.—standard one use and rechargeable and a recharging plug/connection/recharge port 76; (e) a control means 68 push button, switch; (f) at least one capillary and/or sensing lead 80 from the penis to the control and measurement component 70; (g) a display 45 of stroke number and pressure, pulse, blood pressure, calories burned, on the device 30; (h) a wireless connection/blue tooth 77; and (i) a set of options such as a light, LED, sound emitter, 67 wherein the HELU communicates with the wireless connection/blue tooth 77 to a set of personal communication devices 106—such as smart phones, tablets, laptops, personal computers, and the like and then a multitude of potential graph results 108 of body metrics, pressure and number of strokes, and the like can be evaluated and considered for medical analysis and physical status of the individual, for progress toward goals, for games or competitive contests, or merely for novelty and personal amusement.

FIGS. 7A and 7B are sketches of the HELU device 30 in use. These demonstrate: a body metrics, sexual performance and erectile performance analyzing device 30 for measurement monitoring, and testing metrics of an individual; a display 45 of stroke number and pressure, pulse, blood pressure, calories burned, etc.; an expanded portion 46 for controls and display 45; a secondary controls and measurement instruments 60 (accelerometers, sensors, counters, analyzer, counters, etc.); an encasement 62 for controls, instrument, batteries, containers, capsule; a control and measurement component 70 such as an accelerometer, sensors, pedometer like instrument Fit-Bit RTM electronics or the like; a means of power 75 battery, capacitor, LI-ION, NI-CD, alkaline, nickel metal hydride (NIMH), cobalt, rare earth and composite materials etc.—standard one use and rechargeable; wireless connection/blue tooth 77; a capillary and or electrode like sensing leads 80; a person 100 for measurement; a set of personal communication devices 106—such as smart phones, tablets, laptops, personal computers, and the like; a multitude of potential graph results 108 of body metrics, pressure, and number of strokes, and the like; and a penis 110 to be monitored.

The Special Electronic Monitoring Device 30 called HELU which Measures Body Metrics like Pulse, Heart Rates, and Blood Pressure at rest, and which creates a well-defined performance measurement that is used to monitor, analyze and optimize all characteristics of Sexual Activity and Erectile Dysfunction operates as follows: As one can see, the sexual performance and body metrics of a human is measured and monitored by the HELU device 30. The signals from the penis 110 travels through the electrodes or capillaries 80 (depending on the type of pick-up signal needed by the specific instrumentation) to the monitoring electronics 70. The Bluetooth/wireless 77 signal transmits the data to the personal communication devices 106—such as smart phones, tablets, laptops, personal computers, and the like. Then the software and smart phone applications transform the data and send it to a graph or data display 108. These results can be considered for medical analysis and physical status of the individual, progress toward goals, games, or competitive contests, or merely for novelty and personal amusement.

FIGS. 8A through 8E are sketches of an alternative embodiment 32 of a body metrics, sexual performance, and erectile performance analyzing device 30 for measurement, monitoring, and recording body metrics such as blood pressure, calories burned, heart rate, number of strokes and pressure at ejaculation, pulse with features provided. The operation is similar to the above. Once the batteries are charged, the person 100 places the device 32 around the penis and inserts the adjustable end 82 and beads 83 into the aperture 85 of the fixed end 84. Next, press the control means/power button 68 and perform. The data is sent via the wireless transmitter to the personal communications devices 106 and the data can be compared, displayed, graphed, and analyzed. The body metrics can also be measured at rest for a baseline for comparisons of performance data and as well as for medical observations.

Many body metrics and features are contemplated to be measured by the Special Electronic Monitoring Device 30 called HELU which Measures Body Metrics like Pulse, Heart Rates, and Blood Pressure at rest, and which creates a well-defined performance measurement that is used to monitor, analyze and optimize all characteristics of Sexual Activity and Erectile Dysfunction. Some examples, and not limitations, are shown in the following Table.

| ITEM | FEATURES |
|---|---|
| 1 | Erections frequency and duration/ |
| | Erectile Dysfunction/ED indicators |
| 2 | PEDOMETER - strokes before ejaculation; |
| | Stroke Pressures continuously monitored; |
| | maximum pressure achieved at ejaculation; |
| 3 | HEART RATE |
| 4 | BLOOD PRESSURE |
| 5 | BLOOD OXYGEN MEASURING TOOL |
| 6 | TEMPERATURE |
| 7 | STRESS MONITOR |
| 8 | CALORIE COUNTER |
| 9 | VIBRATOR |
| 10 | OPTIONAL SCROTUM LOOP |

With this description it is to be understood that the Special Electronic Monitoring Device 30 called HELU is not to be limited to only the disclosed embodiment of product. The features of the electronic device 30 called a HELU are intended to cover various modifications and equivalent arrangements included within the spirit and scope of the detailed description and operation shown within this specification and illustrated by the accompanying drawings.

While certain novel features of this invention have been shown and described and are pointed out in the annexed claims, it is not intended to be limited to the details above, since it will be understood that various omissions, modifications, substitutions and changes in the forms and details of the device illustrated and in its operation can be made by those skilled in the art without departing in any way from the spirit of the present invention. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

Unless defined otherwise, all technical and scientific terms used herein have the same meaning as commonly understood by one of ordinary skill in the art to which these inventions belong. Although any methods and materials similar or equivalent to those described herein can also be used in the practice or testing of the present inventions, the preferred methods and materials are now described above in the foregoing paragraphs.

Other embodiments of the invention are possible. Although the description above contains much specificity, these should not be construed as limiting the scope of the invention, but as merely providing illustrations of some of the presently preferred embodiments of this invention. It is also contemplated that various combinations or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Thus, it is intended that the scope of at least some of the present inventions herein disclosed should not be limited by the particular disclosed embodiments described above.

The terms recited in the claims should be given their ordinary and customary meaning as determined by reference to relevant entries (e.g., definition of “plane” as a carpenter's tool would not be relevant to the use of the term “plane” when used to refer to an airplane, etc.) in dictionaries (e.g., widely used general reference dictionaries and/or relevant technical dictionaries), commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used herein in a manner more expansive than its ordinary and customary meaning, the term should be given its ordinary and customary meaning plus the additional expansive meaning, or (b) if a term has been explicitly defined to have a different meaning by reciting the term followed by the phrase “as used herein shall mean” or similar language (e.g., “herein this term means,” “as defined herein,” “for the purposes of this disclosure [the term] shall mean,” etc.). References to specific examples, use of “i.e.,” use of the word “invention,” etc., are not meant to invoke exception (b) or otherwise restrict the scope of the recited claim terms. Other than situations where exception (b) applies, nothing contained herein should be considered a disclaimer or disavowal of claim scope. Accordingly, the subject matter recited in the claims is not coextensive with and should not be interpreted to be coextensive with any particular embodiment, feature, or combination of features shown herein. This is true even if only a single embodiment of the particular feature or combination of features is illustrated and described herein. Thus, the appended claims should be read to be given their broadest interpretation in view of the prior art and the ordinary meaning of the claim terms.

Unless otherwise indicated, all numbers or expressions, such as those expressing dimensions, physical characteristics, etc. used in the specification (other than the claims) are understood as modified in all instances by the term “approximately.” At the very least, and not as an attempt to limit the application of the doctrine of equivalents to the claims, each numerical parameter recited in the specification or claims which is modified by the term “approximately” should at least be construed in light of the number of recited significant digits and by applying ordinary rounding techniques.

The present invention contemplates modifications as would occur to those skilled in the art. While the disclosure has been illustrated and described in detail in the figures and the foregoing description, the same is to be considered as illustrative and not restrictive in character, it being understood that only selected embodiments have been shown and described and that all changes, modifications and equivalents that come within the spirit of the disclosures described heretofore and or/defined by the following claims are desired to be protected.

What is claimed is:

1. An Electronic Monitoring Device (30) called HELU wherein the device (30) can be removably secured to a penis (110) of a person/user (100) during Sexual Activity comprising of:
(a) an encasement (62) made of a durable material and having features comprising
(1) at least one pouch (50) for containing a control and measurement component (70),
(3) an elongated strap (63),
(4) a display (45),
(5) a sensor cover (72A),
(6) a control means (68),
(7) a wireless transmitter (77) for remote control, and
(8) a capillary and/or sensor lead (80)
(b) a means of power (75);
(c) a recharging plug/connection/recharge port (76); and
(d) at least one control and measurement component 70;
wherein the HELU communicates with the wireless connection/blue tooth (77) to a set of personal communication devices (106) which provides data for a multitude of potential graph results of body metrics and data on erectile dysfunction (108) that can be evaluated and considered for medical and physical status of the individual, for progress toward goals, for games and competitive contests, and for personal amusement.

2. The Electronic Monitoring Device (30) called HELU described in claim 1 further comprising a second pouch (55) and a second set of capillaries (80).

3. The Electronic Monitoring Device (30) called HELU described in claim 1 wherein the elongated strap is molded into an encapsulating ring of a predetermined size.

4. The Electronic Monitoring Device (30) called HELU described in claim 1 wherein the display (45) further comprises light emitting diodes (LED).

5. The Electronic Monitoring Device (30) called HELU described in claim 1 wherein the control means (68) is selected from the group consisting of a push button, a switch, a relay, and a remote switch.

6. The Electronic Monitoring Device (30) called HELU described in claim 1 wherein the means (40) for removably securing the device (30) to a penis (110) further comprising a Scrotum loop (78).

7. The Electronic Monitoring Device (30) called HELU described in claim 1 further comprising other pouches (55) and a capillary (80) connecting the pouches (50, 55).

8. The Electronic Monitoring Device (30) called HELU described in claim 1 further comprising a light and a sound emitter.

9. The Electronic Monitoring Device (30) called HELU described in claim 1 wherein the means (40) for removably securing the device (30) to a penis (110) further comprises a securing strap (41) with a predetermined inside diameter (66) and an inner surface (72) and an outside diameter (64) and an outer surface (71).

10. The Electronic Monitoring Device (30) called HELU described in claim 1 wherein the durable material for the encasement (62) is selected from the group consisting of silicon, rubber, urethane, composite materials, metals, wood, leather, silk, and synthetic fabrics.

11. The Electronic Monitoring Device (30) called HELU described in claim 10 wherein the metals for the encasement (62) are selected from the group consisting stainless steel, silver, gold, and platinum.

12. The Electronic Monitoring Device (30) called HELU described in claim 1 wherein the means of power (75) is selected from the group consisting of a battery, capacitor, Lithium Ion (LI-ION), nickel cadmium (NI-CD), alkaline, nickel metal hydride (NIMH), cobalt, rare earth, and composite materials.

13. The Electronic Monitoring Device (30) called HELU described in claim 1 wherein the at least one control and measurement component (70) personal communication device (106) is selected from the group consisting of an accelerometer, a sensor, a pedometer, and fitness measuring electronics.

14. The Electronic Monitoring Device (30) called HELU described in claim 1 wherein the personal communication device (106) is selected from the group consisting of smart phones, tablets, laptops, and personal computers.

15. An Electronic Monitoring Device (30) called HELU wherein the device (30) can be removably secured to a penis (110) of a person/user (100) during Sexual Activity comprising of:
(a) an encasement (62) made of a durable material and having features comprising
(1) at least one pouch (50) for containing a control and measurement component (70), at least a second pouch (55) for containing a means of power (75), and a channel/capillary between the first pouch (50) and the second pouch (55).
(3) an elongated strap (63),
(4) a display (45) of body metrics, pressure and number of strokes, calories burned,
(5) a sensor cover (72A),
(6) a push button as a control means (68),
(7) a wireless transmitter (77) for remote control, and
(8) a capillary and/or sensor lead (80)
(b) a means of power (75);
(c) a recharging plug/connection/recharge port (76); and
(d) an accelerometer for stroke pressure instrument, a temperature sensor, and a pedometer as control and measurement components (70)
wherein the HELU communicates with the wireless connection/blue tooth (77) to a set of personal communication devices (106) which provides data for a multitude of potential graph results of body metrics and data on erectile dysfunction (108) that can be evaluated and considered for medical and physical status of the individual, for progress toward goals, for games and competitive contests, and for personal amusement.

16. The Electronic Monitoring Device (30) called HELU described in claim 15 wherein the durable material for the encasement (62) is selected from the group consisting of silicon, rubber, urethane, composite materials, metals, wood, leather, silk, and synthetic fabrics.

17. The Electronic Monitoring Device (30) called HELU described in claim 16 wherein the metals for the encasement (62) are selected from the group consisting stainless steel, silver, gold, and platinum.

18. The Electronic Monitoring Device (30) called HELU described in claim 15 wherein the personal communication device (106) is selected from the group consisting of smart phones, tablets, laptops, and personal computers.

19. The Electronic Monitoring Device (30) called HELU described in claim 15 wherein the means of power (75) is selected from the group consisting of a battery, capacitor, Lithium Ion (LI-ION), nickel cadmium (NI-CD), alkaline, nickel metal hydride (NIMH), cobalt, rare earth, and composite materials.

20. The Electronic Monitoring Device (30) called HELU described in claim 15 further comprising a light and a sound emitter.
